(19) United States
(12) Patent Application Publication
(10) Pub. No.: US 2013/0085464 A1
(43) Pub. Date: Apr. 4, 2013

(54) ABSORBENT ARTICLE AND METHOD OF MANUFACTURING ABSORBENT ARTICLE

(71) Applicants: Jun Kudo, Kagawa (JP); Hideyuki Kinoshita, Kagawa (JP); Yuji Takahashi, Kagawa (JP); Mari Minami, Kagawa (JP)

(72) Inventors: Jun Kudo, Kagawa (JP); Hideyuki Kinoshita, Kagawa (JP); Yuji Takahashi, Kagawa (JP); Mari Minami, Kagawa (JP)

(21) Appl. No.: 13/639,637

(22) PCT Filed: Apr. 6, 2011
(86) PCT No.: PCT/JP2011/059130

§ 371 Date: Dec. 11, 2012

(30) Foreign Application Priority Data
Apr. 6, 2010 (JP) 2010-087989

(51) Int. Cl. A61F 13/15; A61F 13/511

(52) U.S. Cl. CPC A61F 13/15731; A61F 13/51108
USPC 604/380; 264/293

(57) ABSTRACT

An absorbent article in which a longitudinal-direction compression groove is formed along a longitudinal direction of the absorbent article by a compression process performed from the top sheet side, width-direction compression grooves are formed along a width direction of the absorbent article by a compression process performed from the topsheet side, wherein the longitudinal-direction compression groove includes a high compression region, a medium compression region, and a low compression region and the width direction compression groove includes the high compression region and the medium compression region, and the low compression region is disposed over the longitudinal-direction compression groove in the width direction.

### TECHNICAL FIELD

The present invention relates to an absorbent article and a method of manufacturing an absorbent article.

### BACKGROUND ART

Conventionally, there are known absorbent articles having a high compression region and low compression region within a compression groove formed in a laminate portion of a topsheet and an absorber (For example, see Patent Document 1).

In such an absorbent article, the above-described compression groove can be formed without tearing or lifting of the topsheet with the help of a low compression region disposed to protrude out in the width direction from the high compression region.

### CITATION LIST

### Patent Literature

PTL 1: Japanese Unexamined Patent Application Publication No. 2000-14701

### SUMMARY OF INVENTION

### Problem(s) to Be Solved by Invention

However, the applicant has discovered the following problems in the above-described absorbent article.

In the absorbent article, the area of the compression groove increases as the formation of the low compression region on the outer edge of the compression grove, and because the low compression region is disposed in continuation, the compression groove hardens and the rigidity of the entire absorbent article increases.

Particularly, there is a problem that if the rigidity of the compression groove formed along the longitudinal direction of the absorbent article is high, it becomes difficult for the absorbent article to curve in the longitudinal direction; a gap is generated easily between the absorbent article and the wearer during wearing, which can easily cause leakage of body fluids.

Thus, in view of the above problems, an object of the present invention is to provide an absorbent article, which can appropriately maintain the rigidity of a compression groove without tearing and lifting a topsheet, and a method of manufacturing an absorbent article.

### Means for Solving the Invention

The first feature of the present invention is summarized in that an absorbent article including a liquid-permeable topsheet, a liquid-impermeable backsheet, and an absorber disposed between the topsheet and the backsheet, including: a longitudinal-direction compression groove formed along a longitudinal direction of the absorbent article by a compression process performed from the topsheet side; and a width-direction compression groove formed along a width direction of the absorbent article by a compression process performed from the topsheet side, wherein the longitudinal-direction compression groove includes a high compression region, a medium compression region, and a low compression region and the width direction compression groove includes the high compression region and the medium compression region, and the low compression region is disposed over the longitudinal-direction compression groove in the width direction.

The second feature of the present invention is summarized in that a method of manufacturing an absorbent article including a liquid-permeable topsheet, a liquid-impermeable backsheet, and an absorber disposed between the topsheet and the backsheet, the method including forming a compression groove by way of a compression process using a pressure roll from a topsheet side of the absorbent article, wherein the pressure roll is configured such that: a molded protrusion is provided on the outer circumference of the pressure roll, on a contact surface of the absorbent article of the molded protrusion, a plurality of convex units and a plurality of concave units are provided along a rotation direction of the pressure roll, and the concave units are disposed on the edge in the width direction of the molded protrusion.

### Advantageous Effect(s) of Invention

As described above, according to the present invention, it is possible to provide an absorbent article, which can appropriately maintain the rigidity of a compression groove without tearing and lifting a topsheet, and a method of manufacturing an absorbent article.

### BRIEF DESCRIPTION OF DRAWINGS

FIG. 1 is a plan view in which an absorbent article according to a first embodiment of the present invention is seen from a skin contact surface side.

FIG. 2 is a cross-sectional view along line a-a in a plan view in which the absorbent article according to the first embodiment of the present invention is seen from the skin contact surface side.

FIG. 3 is a cross-sectional view along a line b-b in a plan view in which the absorbent article according to the first embodiment of the present invention is seen from the skin contact surface side.

FIG. 4 is a diagram explaining a method of manufacturing an absorbent article according to the first embodiment of the present invention.

FIG. 5 is a diagram explaining the method of manufacturing the absorbent article according to the first embodiment of the present invention.

FIG. 6 is a diagram explaining a pressure roll used in the method of manufacturing an absorbent article according to the first embodiment of the present invention.

FIG. 7 is a diagram explaining the pressure roll used in the method of manufacturing an absorbent article according to the first embodiment of the present invention.

FIG. 8 is a diagram explaining the pressure roll used in the method of manufacturing an absorbent article according to the first embodiment of the present invention.

FIG. 9 is a diagram explaining the pressure roll used in the method of manufacturing an absorbent article according to the first embodiment of the present invention.

FIG. 10 is a diagram explaining a pressure roll used in the method of manufacturing an absorbent article according to a first modification of the present invention.

### DESCRIPTION OF EMBODIMENTS

(First Embodiment of the Present Invention)

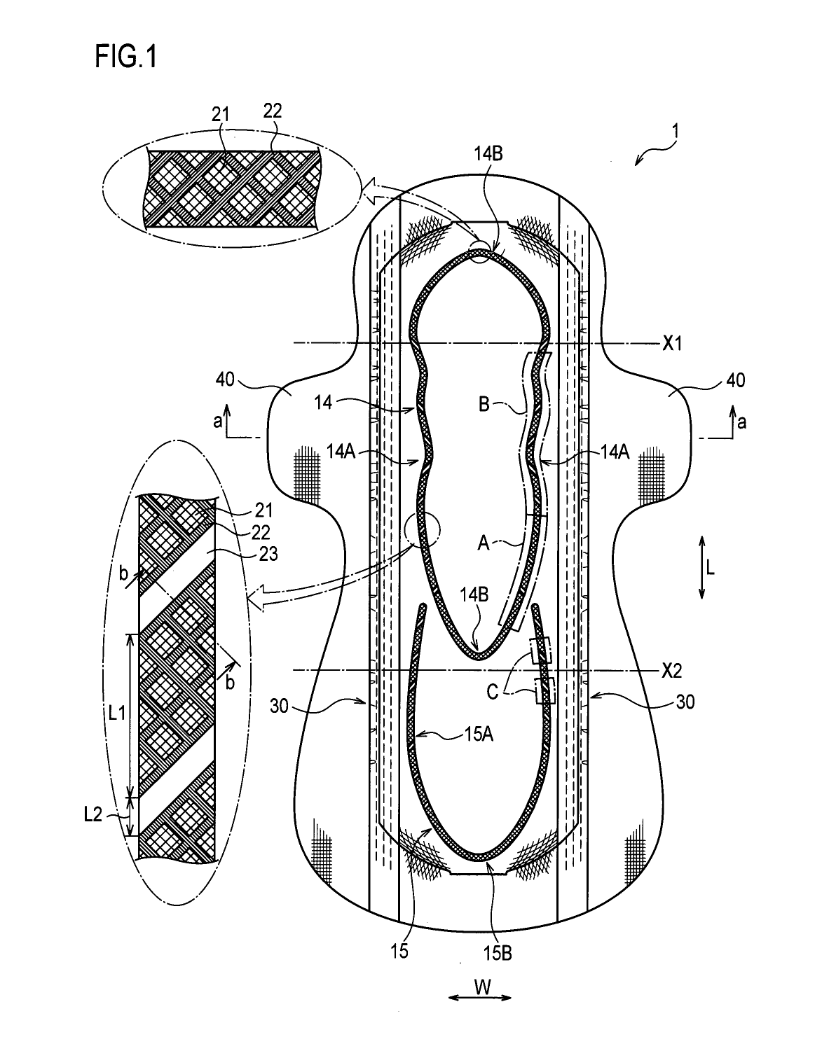

With reference to FIG. 1 to FIG. 9, an absorbent article 1 according to a first embodiment of the present invention will be described. For example, the absorbent article 1 according to this embodiment is a paper diaper, an incontinent pad, a sanitary napkin, etc.

As shown in FIG. 1, the absorbent article 1 according to the embodiment includes a plurality of compression grooves 14 and 15.

Herein, in the absorbent article 1 according to the embodiment, longitudinal-direction compression grooves 14A and 15A formed along a longitudinal direction L by compression process performed from a skin contact surface (topsheet 12) side, and width-direction compression grooves 14B and 15B formed along a width direction W by a compression process performed from the skin contact surface (topsheet 12) side are provided. Here, as shown in FIG. 1, longitudinal-direction compression grooves 14A and 15A may be formed to be diagonal with respect to a straight line along the longitudinal direction L, and an angle (acute angle side) formed by the straight line along the longitudinal direction L and the longitudinal-direction compression groove 14A (15A) is 45 degrees or less. On the other hand, the width-direction compression grooves 14B and 15B may be formed to be diagonal with respect to a straight line along the width direction W, and an angle (acute angle side) formed by the straight line along the width direction W and the width-direction compression groove 14B (15B) is less than 45 degrees.

In this case, as shown in FIG. 1, the longitudinal-direction compression groove 14A and the width-direction compression groove 14B may be connected to form a circular compression groove, or may not be connected. Also, as shown in FIG. 1, the longitudinal-direction compression groove 15A and the width-direction compression groove 15B may be connected to form an approximate U-shaped compression groove, or may not be connected.

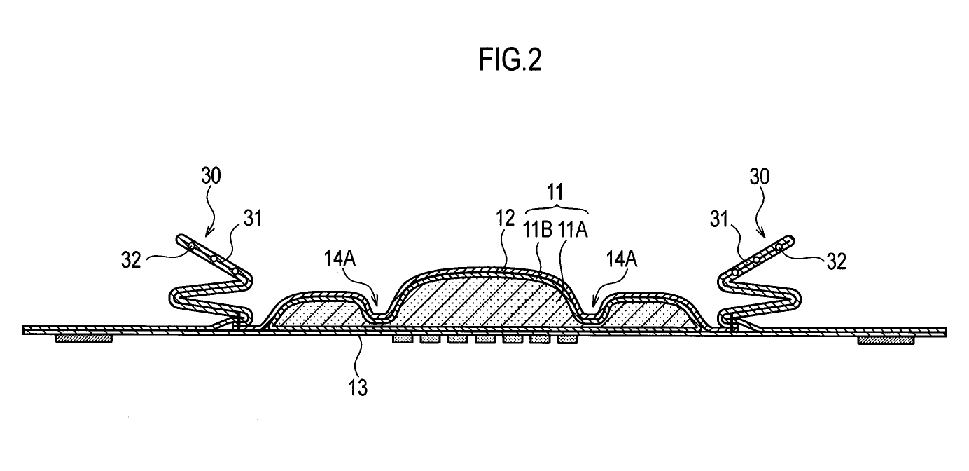

As shown in FIG. 2, the absorbent article 1 according to the embodiment includes a liquid-permeable topsheet 12, a liquid- impermeable backsheet 13, and an absorber 11 disposed between the topsheet 12 and backsheet 13. Conventional materials may be used for the topsheet 12, the backsheet 13, and the absorber 11. For example, a material of the topsheet 12 is not particularly limited as long as it is a sheet material with a liquid-permeable structure, such as a nonwoven fabric, a woven fabric, a porous plastic sheet, a mesh sheet, or the like. Any natural fiber and chemical fiber may be used as a material of the woven fabric and the nonwoven fabric. The backsheet 13 may be a film mainly made of polyethylene, polypropylene or the like, an air-permeable resin film, or a sheet obtained by joining an air-permeable resin film with a nonwoven fiber such as spun bond or spunlace. The absorber 11 may be formed by laminating a hydrophilic fiber or powder by an air-laid method, or may be an air-laid sheet which is formed in such a manner that a hydrophilic fiber or powder is formed to have a sheet shape by the air-laid method.

Furthermore, as shown in FIG. 1 and FIG. 2, the absorbent article 1 according to the embodiment has a leakage-preventing wall 30 on the outer sides of the widthwise-direction W of the absorber 11, which includes a leakage-preventing sheet 31 and an elastic member 32.

As shown in FIG. 1, the longitudinal-direction compression groove 14A includes a high compression region 21, a medium compression region 22, and a low compression region 23, and the width-direction compression groove 14B includes a high compression region 21 and a medium compression region 22.

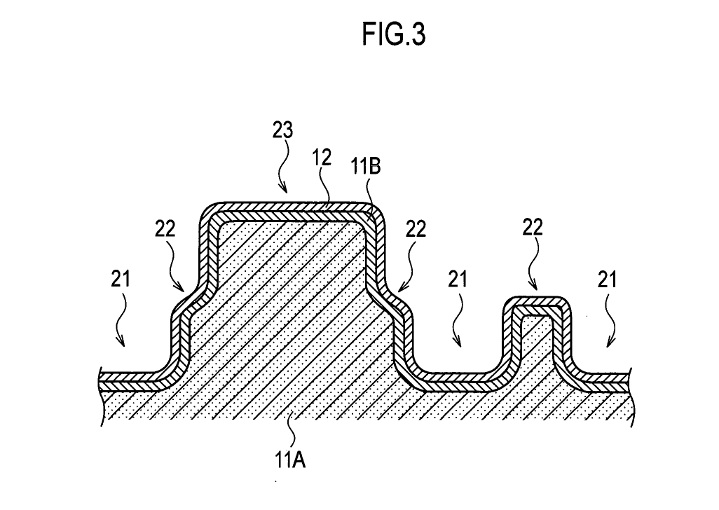

Herein, as shown in FIG. 3, the thickness of the high compression region 21 is smaller than the thickness of the medium compression region 22, and thickness of the medium compression region 22 is smaller than the thickness of the low compression region 23.

The thickness was measured with the following measurement method. Specifically, in this measurement method, after freezing the sample absorbent article 1 by immersing it in liquid nitrogen, it was cut with a blade, returned to normal temperature, and then measured at 50-fold magnification by using an electronic microscope (such as Keyence-make VE7800). Here, the reason for freezing the sample absorbent article 1 is to prevent variation in the thickness due to compression during cutting.

For example, in the longitudinal direction L and the width direction W, the high compression region 21 may be disposed between the medium compression regions 22. As shown in the example in FIG. 1, in the longitudinal direction L and the width direction W, the high compression regions 21 are disposed between the medium compression regions 22 that are disposed in a lattice.

Furthermore, in the longitudinal direction L, the medium compression regions 22 may be disposed between the low compression regions 23. As shown in the example in FIG. 1, in the longitudinal direction L, the medium compression regions 22 are disposed between the low compression regions 23 disposed over the longitudinal-direction compression groove 14A in the width direction W.

Thus, according to the configuration in which the medium compression regions 22 are disposed between the low compression regions 23, and the high compression regions 21 are disposed between the medium compression regions 22, through gradual compression inside the longitudinal-direction compression groove 14A, a difference in elevation in the longitudinal-direction compression groove 14A is reduced, and tearing the topsheet 12 inside the longitudinal-direction compression groove 14A can be prevented.

Furthermore, the absorbent article 1 according to the embodiment is packed individually by folding along a plurality of folding lines in the width direction W. For example, in the embodiment, as shown in FIG. 1, the absorbent article 1 is packed individually by folding into three along two folding lines X1 and X2.

Here, the low compression region 23 is disposed in a region A inside the longitudinal-direction compression groove 14A. The region A is a region between a base portion of a pair of wing portions 40 formed to protrude on both outer sides of the width direction W and the folding line X2 on the posterior side of the longitudinal direction L, and is a region that curves the most while wearing.

Further, the low compression region 23 is disposed in the region B inside the longitudinal-direction compression groove 14A. The region B is an excretion-portion contact region that contacts a vaginal opening of a wearer in the absorbent article 1 and that is disposed between two leg openings of an underwear when the absorbent article 1 is worn on the underwear of the wearer. In the example shown in FIG. 1, the region B is provided in a longitudinal-direction zone where the pair of wing portions 40 is provided.

Thus, because the region B is large in weight of the absorber 11, by providing the low compression region 23 in the region B, the rigidity of the region B can be reduced.

Furthermore, the low compression region 23 may be disposed in a region C inside the longitudinal-direction compression groove 15A. The region C is a region that is close to the folding line X2 in the longitudinal direction L.

Here, because the rigidity declines at the folding line X2 in the longitudinal direction L, by providing the low compression region 23 in the region C, which is the region close to the folding line X2, more specifically, which is the region adjacent to the folding line X2, a difference in the rigidity between the region C and the region of the folding line X2 can be reduced.

As a result, because the absorbent article 1 easily curves at locations other than the folding line X2 (that is, the region C), the gap that is formed by the folding line X2 when the absorbent article 1 is worn can be reduced.

Inside the longitudinal-direction compression groove 14A, the area occupied by the high compression region 21 and the medium compression region 22 may be larger than the area occupied by the low compression region 23.

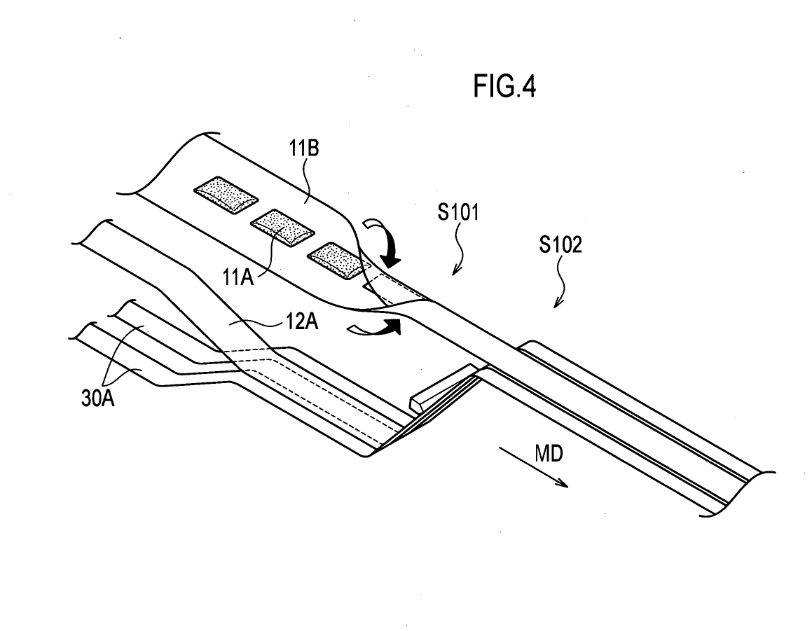

For example, as shown in FIG. 1, in the longitudinal direction L, the length L 1 of the region in which the high compression region 21 and the medium compression region 22 are provided is longer than the length L2 of the region in which the low compression region 23 is provided. Next, one part of a method of manufacturing the absorbent article 1 according to the embodiment is described with reference to FIG. 4 to FIG. 9. It is noted that as far as the methods that are not described in FIG. 4 to FIG. 9 are concerned, the existing methods can be used.

As shown in FIG. 4, in step S101, the absorber 11 is produced by wrapping a laminated body 11A made of ground pulp and absorber polymer with a tissue 11B.

In step S102, the absorber 11 is disposed on top of a web 12A that is conveyed continuously along with the machine direction MD and is used for the topsheet 12 on which a leakage-preventing wall sheets 30A are pasted, and the web 12A used for the topsheet 12 and the absorber 11 are pasted together with a hot-melt adhesive.

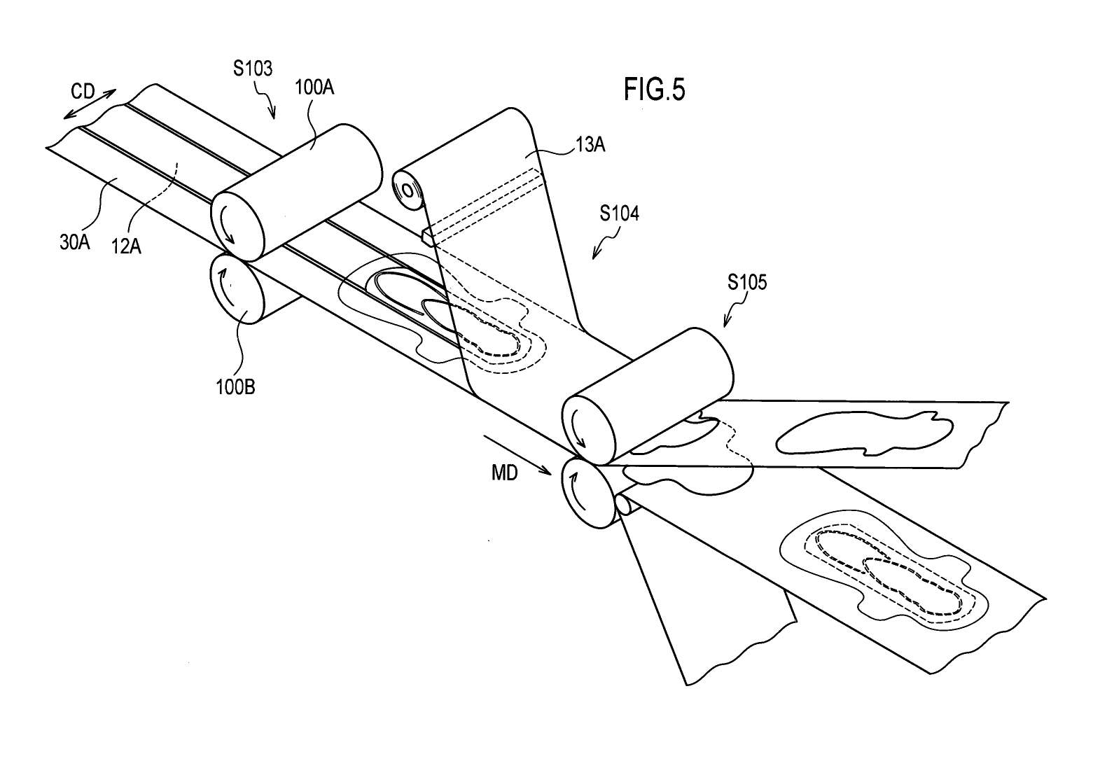

As shown in FIG. 5, in step S103, the longitudinal-direction compression grooves 14A, 15A and the width-direction compression grooves 14B, 15B are formed in the leakage-preventing wall sheet 30A, the web 12A used for topsheet 12, and the absorber 11 that are pasted together by performing the compression process using pressure rolls 100A and 100B from the side of the web 12A for the topsheet 12.

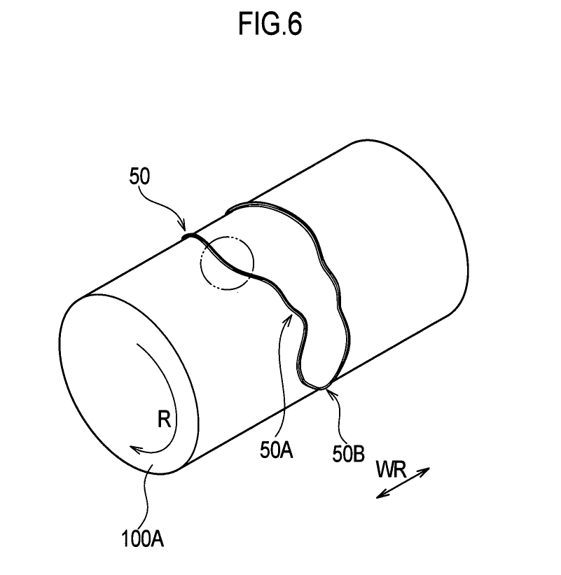

As shown in FIG. 6, a molded protrusion 50 is formed on the outer circumference of the pressure roll 100A.

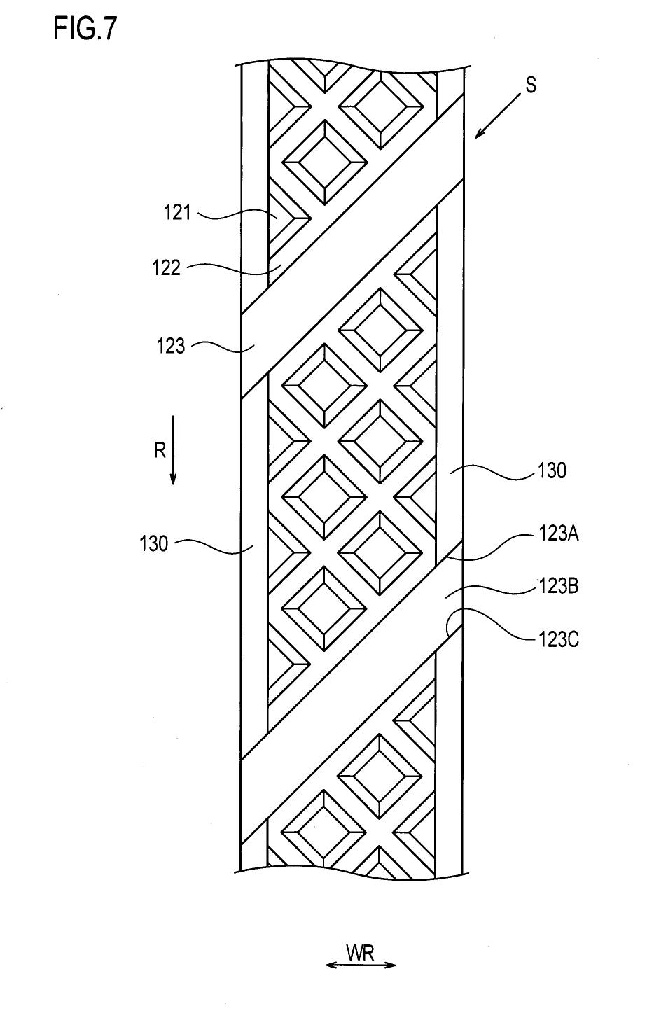

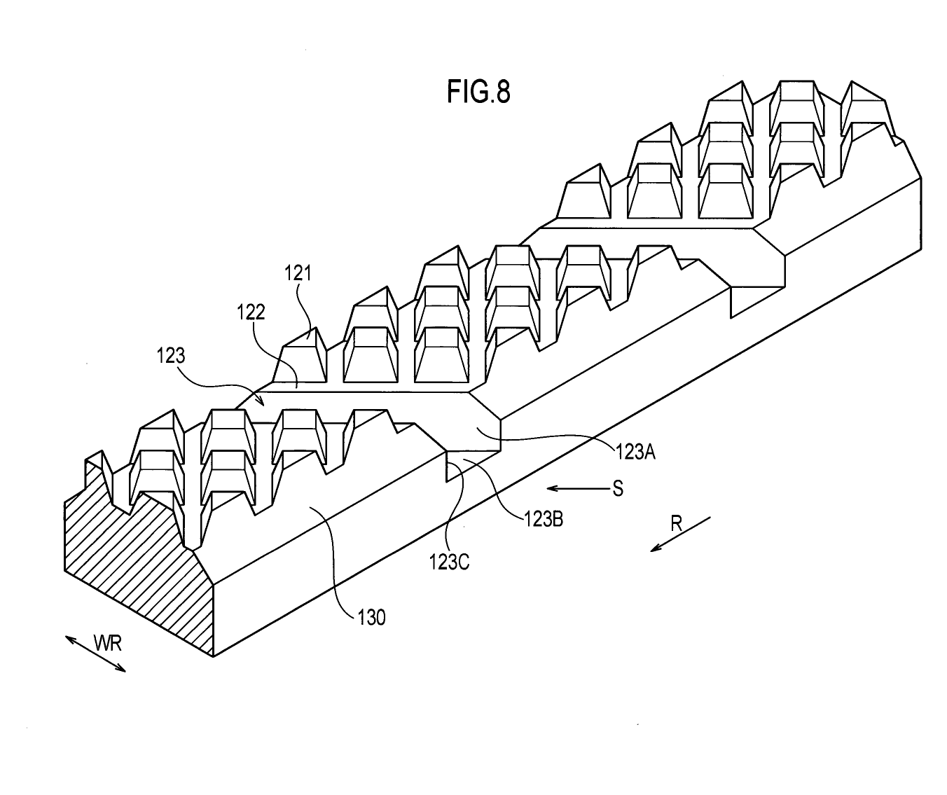

As shown in FIG. 7 and FIG. 8, on the contact surface 122 of the absorbent article 1 of the molded protrusion 50, a plurality of convex units 121 and a plurality of concave units 123 are formed along a rotation direction R of the pressure roll 100A.

Further, in the molded protrusion 50, a sidewall surface 130 is provided outside the width direction WR of the pressure roll 100A of the contact surface 122, i.e., on the edge of the width direction WR.

As shown in FIG. 8, the sidewall surface 130 is provided as an inclined surface. The sidewall surface 130 may be provided as a flat surface. The sidewall surface 130 may be provided either on both-side edges of the width direction WR or only on a single-side edge of the width direction WR. Here, the concave units 123 are disposed on the edge of the width direction WR, i.e., on the sidewall surface 130.

For example, the convex units 121 may be disposed between the contact surfaces 122 in the rotation direction R and the width direction WR. As shown in the examples in FIG. 7 and FIG. 8, the convex units 121 may be disposed between the contact surfaces 122 disposed in a lattice in the rotation direction R and the width direction WR.

Furthermore, the contact surface 122 may be disposed between the concave units 123 in the rotation direction R. As shown in the examples in FIG. 7 and FIG. 8, the contact surface 122 may be disposed in the rotation direction R between the concave units 123 disposed over the molded protrusion 50 in the width direction WR.

As shown in the examples in FIG. 7 and FIG. 8, the concave units 123 may be disposed between the convex units 121 in the rotation direction R. In other words, the convex units 121 and the concave units 123 may be disposed in an approximate alternate pattern in the rotation direction R.

The molded protrusion 50 is configured by a rotational-direction molded protrusion 50A provided along the rotation direction R and a width-direction molded protrusion 50B provided along the width direction WR of the pressure rolls 100A and 100B. The rotational-direction molded protrusion 50A forms longitudinal-direction compression grooves 14A and 15A, and the width-direction molded protrusion 50B forms the width-direction compression grooves 14B and 15B. That is, in the rotational-direction molded protrusion 50A, the angle (acute angle side) formed by a straight line orthogonal to the width direction WR and the rotational-direction molded protrusion 50A is 45 degrees or less. On the other hand, in the width-direction molded protrusion 50B, an angle (acute angle side) formed by a straight line along the width-direction WR and the width-direction molded protrusion 50B is less than 45 degrees.

Here, the concave units 123 are provided on the contact surface 122 of the absorbent article 1 in the rotational-direction molded protrusion 50A, but may not be provided on the contact surface 122 of the absorbent article 1 in the width-direction molded protrusion 50B. Here, the molded protrusion 50B comprise a passage extending from the transversal center towards a side edge of the pressure roll 100A.

The molded protrusion 50 is configured such that on the contact surface 122, the area occupied by the region in which the concave units 123 are provided is smaller than the area occupied by the other regions (that is, the region in which the convex units 121 are provided and the region in which the convex units 121 and concave units 123 are not provided).

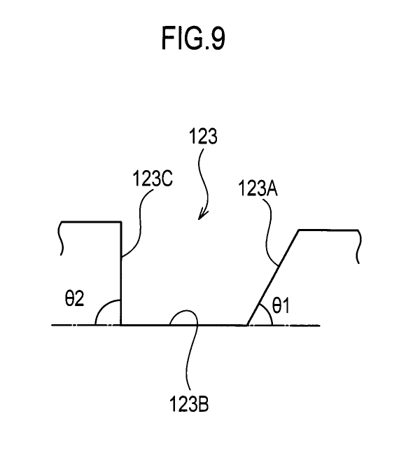

As shown in FIG. 9, in the molded protrusion 50, an angle θ1 formed by the side surface 123A of the concave units 123 and the bottom surface 123B of the concave units 123 in the anterior side of the rotation direction R may be between 15 and 60 degrees.

On the other hand, in the molded protrusion 50, an angle θ2 formed by the side surface 123C of the concave units 123 and the bottom surface 123B of the concave units 123 in the posterior side of the rotation direction R may be either an approximate right angle or between 15 and 60 degrees.

According to the configuration, during the compression process, it is possible to accomplish an effect that it is difficult to tear the web 12A used for the topsheet 12.

It is noted that FIG. 9 is a diagram of the molded protrusion 50 shown in FIG. 7 and FIG. 8 as seen from the direction of an arrow S.

In step S104, the web 13A used for the backsheet 13 is pasted onto the leakage-preventing wall sheet 30A, the web 12A, and the absorber 11 used for the topsheet 12 for which the compression process has been performed.

In step S105, the leakage-preventing wall sheets 30A, the web 12A used for the topsheet 12, the absorber 11, and the web 13A used for the backsheet 13 that have been pasted together are cut into a predetermined size and shape by a product cutter to form the absorbent article 1.

According to the embodiment-based absorbent article 1, by providing the high compression region 21, the medium compression region 22, and the low compression region 23, it becomes easy for the absorbent article 1 to curve in the longitudinal direction L in the low compression region 23 and flexibly follow along the body curves of the wearer, and therefore, a situation giving rise to a gap between the wearer and the absorbent article 1 during wearing, which causes leakage of body fluids, can be reduced.

According to the embodiment-based absorbent article 1, because the low compression region 23 is not provided in the width-direction compression groove 14B, the durability is possessed against pressure from a groin of the wearer, and therefore, reduction of the surface that absorbs the body fluids becomes difficult, and thereby, leakage of body fluids can be reduced.

According to the embodiment-based absorbent article 1, the area occupied by the high compression region 21 and the medium compression region 22 is larger than the area occupied by the low compression region 23, and therefore, the generation of a twist caused as a result of an excessive decline in the rigidity of the absorbent article 1 can be prevented.

According to the embodiment-based method of manufacturing the absorbent article 1, with the help of the concave units 123 formed by cutting out a part of the side wall surface 130 of the protrusion 50 of the pressure roll 100A, it is possible to form the low compression region 23 that enables a decline in the rigidity in the longitudinal direction L of the absorbent article 1 without tearing the web 12A used for the topsheet 12.

(First Modification)

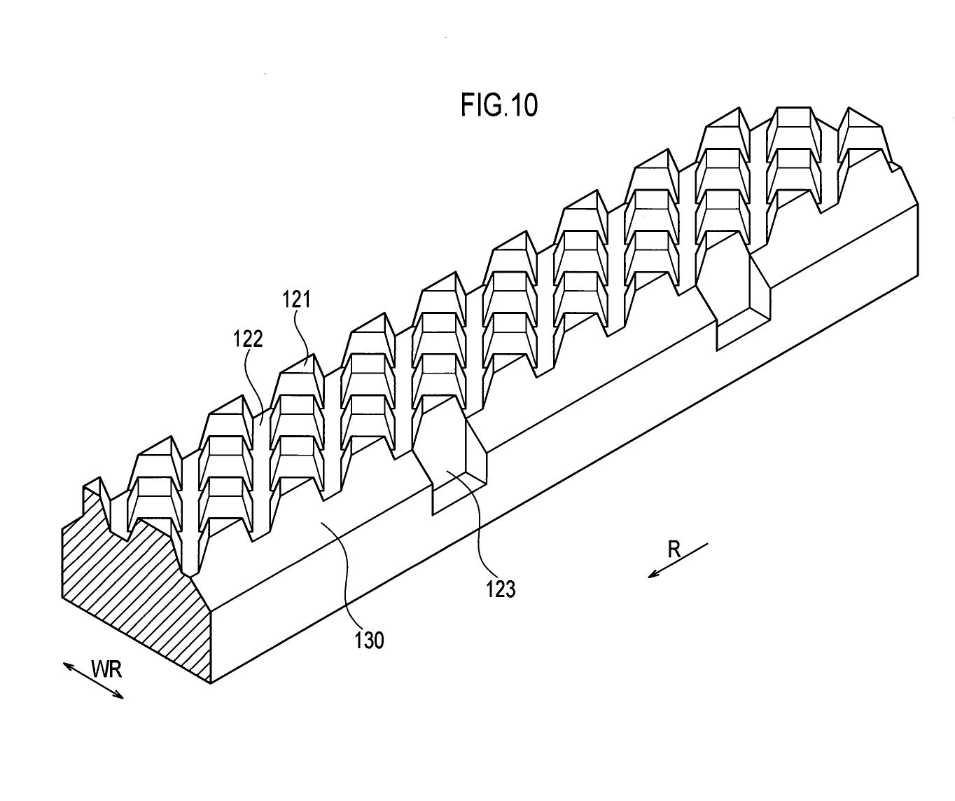

With reference to FIG. 10, a method of manufacturing the absorbent article 1 according to a first modification of the present invention will be explained. Hereinafter, the method of manufacturing the absorbent article 1 according to the first modification will be explained with an emphasis on a difference from a method of manufacturing the absorbent article 1 according to the above-described first embodiment.

As shown in FIG. 10, in the pressure roll 100A used in the method of manufacturing the absorbent article 1 according to the first modification, the concave units 123 are not disposed over the molded protrusions 50 in the width direction WR. For example, as shown in FIG. 10, the concave units 123 may be disposed across the entire sidewall surface 130 in the width direction WR, or may be disposed across a part of the sidewall surface 130.

Also, as shown in FIG. 10, in the pressure roll 100A used in the method of manufacturing the absorbent article 1 according to the first modification, the concave units 123 are not disposed between the convex units 121 in the rotation direction R. For example, as shown in FIG. 10, the concave units 123 may be disposed at every predetermined interval to overlap at least one part of the portion in the convex units 121 in the rotation direction R.

Thus, the present invention has been explained in detail by using the above-described embodiments; however, ithe present invention is not limited to the embodiments explained herein. The present invention can be implemented as a corrected, modified mode without departing from the gist and the scope of the present invention defined by the claims. Therefore, the description of the specification is intended for explaining the example only and does not impose any limited meaning to the present invention.

Note that this application claims the benefit of Japanese Application No. 2010-087989 the entire disclosure of which is incorporated by reference herein.

1. An absorbent article comprising:
a liquid-permeable topsheet;
a liquid-impermeable backsheet;
an absorber disposed between the topsheet and the backsheet;
a longitudinal-direction compression groove formed along a longitudinal direction of the absorbent article by a compression process performed from the topsheet side; and
a width-direction compression groove formed along a width direction of the absorbent article by a compression process performed from the topsheet side, wherein
the longitudinal-direction compression groove includes a high compression region, a medium compression region, and a low compression region and the width direction compression groove includes the high compression region and the medium compression region, and
the low compression region is disposed over the longitudinal-direction compression groove in the width direction.

2. The absorbent article according to claim 1, wherein in the longitudinal-direction compression groove, the high compression region is disposed between the medium compression regions in the longitudinal direction and the width direction, and the medium compression region is disposed between the low compression regions in the longitudinal direction.

3. The absorbent article according to claim 1, wherein in the longitudinal-direction compression groove, an area occupied by the high compression region and the medium compression region is larger than an area occupied by the low compression region.

4. A method of manufacturing an absorbent article including a liquid-permeable topsheet, a liquid-impermeable backsheet, and an absorber disposed between the topsheet and the backsheet, the method comprising:
forming a compression groove by way of a compression process using a pressure roll from a topsheet side of the absorbent article, wherein the pressure roll is configured such that:
a molded protrusion is provided on the outer circumference of the pressure roll, on a contact surface of the absorbent article of the molded protrusion, a plurality of convex units and a plurality of concave units are provided along a rotation direction of the pressure roll, and
the concave units are disposed on the edge in the width direction of the molded protrusion.

5. The method of manufacturing an absorbent article according to claim 4, wherein in the rotation direction, the concave units are disposed between the convex units.

6. The method of manufacturing an absorbent article according to claim 4, wherein the concave units are disposed over the contact surface in the width direction.

7. The method of manufacturing an absorbent article according to claim 4, wherein
the molded protrusion is configured by a rotation-direction molded protrusion provided along the rotation direction and a width direction molded protrusion provided along the width direction of the pressure roll, and
the concave units are provided on the contact surface of the absorbent article in the rotation-direction molded protrusion, and are not provided on the contact surface of the absorbent article in the width direction molded protrusion.

8. The method of manufacturing an absorbent article according to claim 4, wherein an angle formed by a side surface of the concave unit and a bottom surface of the concave unit in an anterior side of the rotation direction is between 15 and 60 degrees.
